(19) United States
(12) Patent Application Publication
(10) Pub. No.: US 2024/0001136 A1
(43) Pub. Date: Jan. 4, 2024

(54) ANGLED TRANSCRANIAL MAGNETIC STIMULATION DEVICE

(71) Applicants: University of Maryland, Baltimore County, Baltimore, MD (US); University of Maryland, Baltimore, Baltimore, MD (US)

(72) Inventors: Fow-Sen CHOA, Baltimore, MD (US); L. Elliott Hong, Baltimore, MD (US)

(21) Appl. No.: 18/345,614

(22) Filed: Jun. 30, 2023

Related U.S. Application Data
(60) Provisional application No. 63/357,231, filed on Jun. 30, 2022.

(51) Int. Cl. A61N 2/02; H01F 27/28; A61N 2/00

(52) U.S. Cl. CPC A61N 2/02; A61N 2/006; H01F 27/2823

(57) ABSTRACT

The present invention relates to angle-tuned (AT) ring coil devices to reduce the individual coil footprint and improve depth-spread characteristics of transcranial magnetic stimulation (TMS) systems. The AT coil device includes multiple stacked coils, which enhances field strength, reduces the footprint, and increases the field penetration depth by modifying its geometric distribution. Moreover, the AT coil devices demonstrated superior performance for multisite stimulation due to their smaller footprint, making them suitable for multisite stimulations of inter and intra-hemispheric brain regions with an improved spread and less electric field divergence.

### CROSS REFERENCE TO RELATED APPLICATIONS

This application claims priority to U.S. Provisional Patent Application No. 63/357,231 filed on Jun. 30, 2022 in the name of L. Elliott HONG and Fow-Sen CHOA entitled “ANGLED TRANSCRANIAL MAGNETIC STIMULATION DEVICE,” which is hereby incorporated by reference herein in its entirety.

### STATEMENT OF FEDERALLY SPONSORED RESEARCH

This invention was made with government support under Grant No. 1631820 awarded by the National Science Foundation. The government has certain rights in the invention.

### FIELD

The present invention relates to an angle-tuned ring coil for improving the depth-spread performance of transcranial magnetic stimulation (TMS) coils as well as high-performance composite coils and multisite TMS systems.

### BACKGROUND

Transcranial magnetic stimulation (TMS) is a rapidly evolving non-invasive neuromodulation technique and an established U.S. Food and Drug Administration (FDA) treatment for major depression disorder, migraine, and obsessive-compulsive disorder [1, 2]. Recently smoking addiction has also been accepted as FDA approved TMS treatment items. The applications of TMS have been further extended to areas that cover brain connectivity, cognitive, perceptual, behavioral, and therapeutic investigations, and treatment [3-5]. Since normal and pathological brain functions involve multiple brain networks, and each brain network contains multiple sub-regions [6, 7], tools like dual-coil TMS can provide exceptional opportunities to investigate effective connectivity and plasticity through the ability to utilize excitatory or inhibitory stimulations to change long-term potentiation and long-term depression of interconnected brain regions [8-12].

Multisite neuromodulation with controlled timing provides a tool for mechanistic studies of coordinated brain dynamics, complex gating effect in humans, and validating brain connectivity biomarkers, in addition to the treatment of neurologic and psychiatric disorders [13-16]. However, conventional circular and figure-8 TMS coils occupy a substantial footprint, defined here as the tangential surface area the coil occupies in the contact surface plane closest to the head. Further, with current TMS tools, the field diverges quickly and it is difficult to activate deep brain regions, where many neural disorders take place. Therefore, it is challenging to accomplish more than two stimulation sites with the flexibility to move the coils around and reach the desired locations. Due to the large size of the stimulating coils, the multisite stimulation is predominantly focused on inter-hemispheric connectivity between brain regions [17, 18]. This complication cannot be resolved by shrinking the coil size to accommodate the space congestion challenge since the smaller conventional coils have higher field divergence characteristics, preventing them from providing sufficient field intensity for a suprathreshold stimulation at a typical depth for the human cortex.

It is desirable to have a TMS tool that can have less spread for targeting more defined areas and ideally also can reliably target deeper cortical regions. Moreover, animal experiments are often required to advance TMS science, and animal coils with a targeted stimulation typically require spot size down to a few mm scale to precisely target rodent brain targets, further increasing the demand for focality of the TMS coil design. Currently there is no commercial TMS tool available to be able to activate such a focused area.

### SUMMARY

In one aspect, an angled-tuned (AT) transcranial magnetic stimulation (TMS) coil device is described, said AT coil device comprising:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane of the non-metal coil holder.

In another aspect, a TMS system is described, said TMS system comprising:
a mechanical frame; and
at least one AT coil device attached to the mechanical frame for adjustment of the at least one AT coil device,
wherein the at least one AT coil device comprises:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane of the non-metal coil holder.

In still another aspect, a TMS system is described, said TMS system comprising:
a mechanical frame; and
at least two AT coil devices attached to their own dedicated mechanical frame for adjustment of the at least two AT coil devices, wherein the at least two AT coil devices are arranged in a “V shape” or an “A shape,” relative to a horizontal plane,
wherein the at least one AT coil device comprises:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane.

Other aspects, features and embodiments of the invention will be more fully apparent from the ensuing disclosure and appended claims.

### BRIEF DESCRIPTION OF THE FIGURES

FIG. 1A. Illustration of side-view for a single angle-tuned TMS coil with five winding layers located above a homogenous and spherical head model (left) and a top view of a winding layer showing the winding layer inner diameter (ID) and outer diameter (OD).

FIG. 1B. The definitions of the half-value depth (d_{1/2}), half-value volume (V_{1/2}), and half-value spread (S_{1/2}) used for the estimation of the depth-spread characteristics for the coils. The cerebral cortex is defined as 1.5 cm below the surface of the head model. The half-value depth is defined as the radial distance from the cortical surface to the deepest point where the electric field strength is half of the maximum field strength on the cortical surface.

FIG. 1C. A plan view of the definition of the footprint. For the figure-8 coil (shown in blue), the footprint involves two circular coils with an outer diameter of 8.7 cm for each and is around 120 cm² (dotted blue lines). In contrast, for the AT coil (shown in red), the footprint is calculated by multiplying the coil's surface area by the cosine of the tilting angle. The footprint for a single 4.5-cm outer diameter is around 5.5 cm² (dotted red lines).

FIG. 1D. The coil placement schematic for experiments and simulations. Top—the coil and core axis are perpendicular to the measurement cut-plane (head plane). The red lines demonstrate the streamline of the generated magnetic field. Bottom, right—experimentally fabricated coil based on nine winding layers wrapped over a 3D printed coil holder. The alignment of the coil is shown with the coordination axis. The fabricated coil has an inner and outer diameter of 1 cm and 3 cm, respectively, with a tilting angle of 20 degrees. Bottom, left—the FEM simulation model.

FIG. 1E. A schematic of a plan view (left) and a top view (right) of one embodiment of an AT coil holder.

FIG. 2A. Depth-spread performance of the coils. Effect of tilting angle and accumulation of wire wrapped coils on S_{1/2} and d_{1/2}. The circles represent the locations of the previously studied coils by Deng et al. [20]. The solid and dashed lines show the best-fit curves for the circular and figure-8 types of coils, respectively. The red dots are the calibrations performed in this study in COMSOL to validate the simulation technique. For all three cases, the calibration results are within 1% of the previously reported data. The AT coils have an inner and outer diameter of 8 cm and 9 cm, respectively, with tilting angles ranging from 0 to 70 degrees with 10-degree steps and winding layers of 2, 5, and 9. The previously obtained data from Deng et al. are shown in the background for comparison purposes.

FIG. 2B. Effect of the coil's outer diameter on S_{1/2} and d_{1/2}. In the plot, the OD represents the outer diameter of the studied coil. The OD of the single AT coils varies from 2 cm to 100 cm, with the winding width kept constant at 1 cm, meaning that the inner diameter is 1 cm smaller than the outer diameter.

FIG. 2C. Effectiveness of using multiple AT coils as pairs for improving the depth-spread. The composite coil structure, a 4-coil design, is shown in the top left corner, with the red arrows showing each coil's current direction. In this design, two 80-degree tilted coils with an internal angle of 20 degrees form a pair; the coil design includes two of these pairs with opposite polarities and an internal angle of 60 degrees, and the coil OD changes from 4.5 to 30 cm with 1 cm winding width.

FIG. 3A. Multisite stimulation apparatus using AT coils. 4 AT coils used for simultaneous stimulation of different brain regions. A mechanical frame in a ring structure is considered while the coils' location can be adjusted in this frame.

FIG. 3B. Plan view of the apparatus with 4 AT coils. This figure demonstrates the smaller footprint of single AT coils (shown in red) and the 4-coil apparatus compared to the figure-8 coil (shown in blue). The single AT coil's occupied footprint and the figure-8 coils are 5.5 cm² and 120 cm², respectively.

FIG. 3C. Side view of the apparatus with 2 AT coils, arranged in a V-shape. This view demonstrates the capability of the apparatus for stimulating two points as close as 1 cm to each other. Also, the system can be used for complex structures with enhanced depth-spread performance.

FIG. 4A. Electric field distribution for the coils with different tilting angles based on the simulations.

FIG. 4B. A 7 cm-dia (D-70) standard Magstim coil.

FIG. 4C. Measured electric field vector of the coil.

FIG. 4D. Electric field distribution for the coils with different tilting angles based on the experimental measurements. All the measurements were performed in 1.5 cm distance from the surface of the coil with the dark red areas showing the highest intensities encountered; all the data is normalized to the highest electric field intensity. In the experimental data, the scanned area for the coil-B and the figure-8 coil is larger than the coil-A series due to their larger size, which resulted in more pixels for the two coils.

FIG. 5A. Hot spot size for experimental data and simulations.

FIG. 5B. Electric field intensity decay rate (in percentage) as a function of depth for the experimental measurements.

FIG. 5C. Electric field intensity decay rate (in percentage) as a function of depth for FEM simulations.

FIG. 6A. Structure design of an 8 element composite coil that can do 4-site simultaneous stimulations.

FIG. 6B. The measured electric field distribution of the composite coil at 0.8 cm away from the coil.

FIG. 6C. The measured electric field distribution of the composite coil at 2 cm away from the coil.

FIG. 7A. The comparison of depth-spread tradeoff curves for different coil designs.

FIG. 7B. An illustration of the focus obtained from joining multiple pairs of AT coils, as described herein, together with an angle. Each ellipse represents the field distribution created by two AT coil designs arranged in an “A shape.”

FIG. 7C. A plan view of the 8-coil design embodiment described herein. In this design, two 80-degree tilted AT coils with an angle of 20 degrees between them form an “A shape” pair. Two of these pairs with opposite polarities and an internal angle of 60 degrees between them are arranged to form a composite coil, and the coil ID changes from 3.5 to 29 cm with a 1 cm winding width.

FIG. 7D. A top view of the 8-coil design embodiment of FIG. 7C.

### DETAILED DESCRIPTION, AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Subject” as used herein refers to any vertebrate such as mammals, birds, reptiles, amphibians and fish including, but not limited to, a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, monkey, chimpanzee, and humans. In some embodiments, the subject is a human.

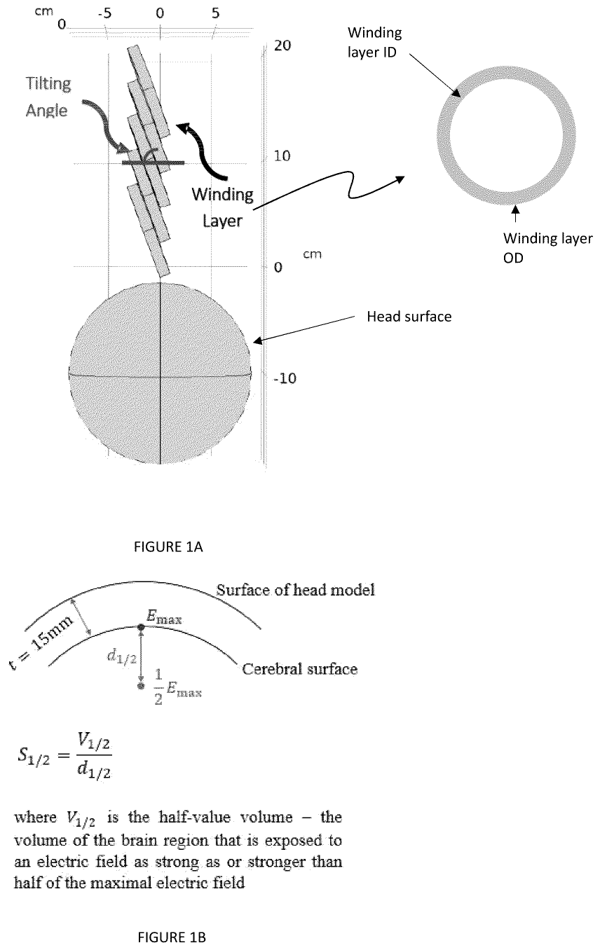

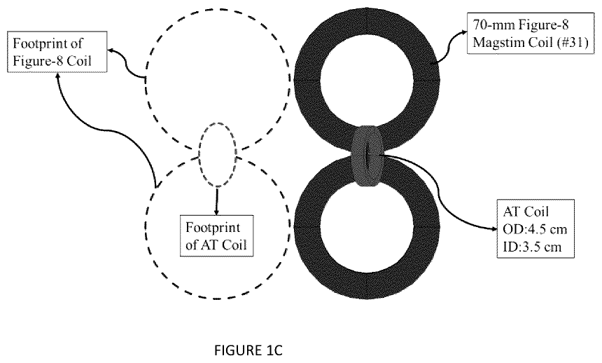

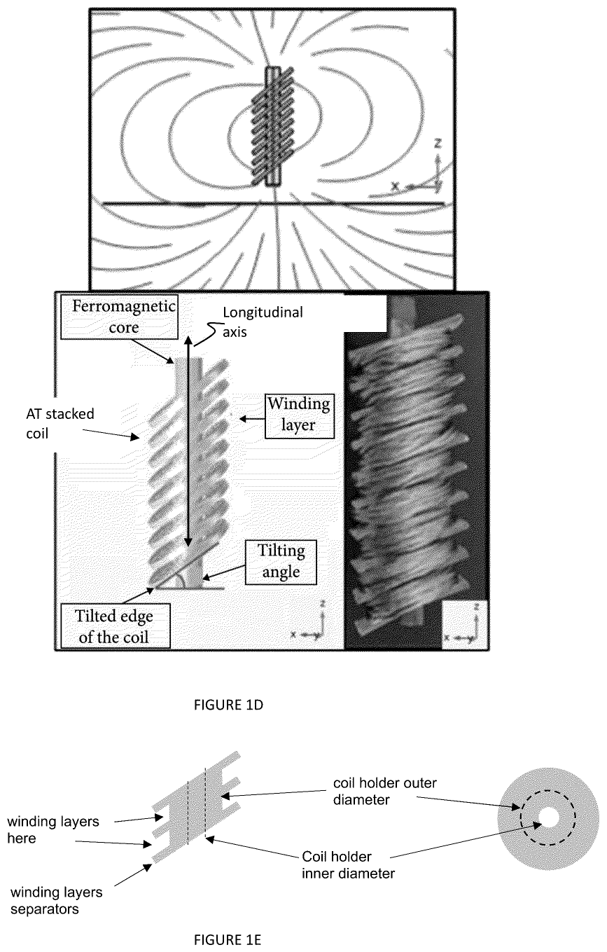

It is understood that the “horizontal plane” is the X-Y plane, as shown for example in FIG. 1D and can be applied to all of the figures including AT coil devices.

Broadly, an angle-tuned TMS device configured to reduce the footprint for TMS systems and allow improved treatments is described. The angle-tuned (AT) TMS device comprises at least one AT coil device, which can have various geometric arrangements (for example can be angled relative to the horizontal plane). AT coil devices can comprise stacked and/or angled layers. The AT coil devices described herein can reduce the individual coil footprint and improve depth-spread characteristics in TMS systems. The field-shaping technique and the structure do not require counter-field generations, making it easy to implement and modify. The AT coil devices comprising stacking layers enhance field strength, reduce the footprint, and increases the field penetration depth by modifying its geometric distribution. By manipulating the coils' composite structure along the Z-direction, a sharper elliptical electric field distribution can be induced and the electric field strength can be enhanced through the superposition of the stacked coils. In some embodiments, increasing the coil wire-wrapping angle reduces the field spread by introducing asymmetry to the coils' structure. These AT coil devices demonstrate better spread, higher electric field penetration, better field decay rate, and smaller footprints than conventional coils, making them suitable for studies on inter- and intra-hemispheric interactions in the brain's neural network.

FIG. 1D shows an embodiment of an AT coil device described herein. In some embodiments, AT coil devices can be fabricated by wrapping wire over 3-D printed coil holders, which can be printed with different angles and dimensions. In some embodiments, the AT coil device has anywhere from 2 to 9 individual winding layers over a 3D printed coil holder. In some embodiments, the AT coil device has 2-4 individual winding layers over the 3D printed coil holder. In some embodiments, the AT coil device has 3-5 individual winding layers over the 3D printed coil holder. In some embodiments, the AT coil device has 4-6 individual winding layers over the 3D printed coil holder. In some embodiments, the AT coil device has 5-7 individual winding layers over the 3D printed coil holder. In some embodiments, the AT coil device has 6-8 individual winding layers over the 3D printed coil holder. In some embodiments, the AT coil device has 7-9 individual winding layers over the 3D printed coil holder. In some embodiments, the AT coil device has 2 individual winding layers over the 3D printed coil holder. In some embodiments, the AT coil device has 3 individual winding layers over the 3D printed coil holder. In some embodiments, the AT coil device has 4 individual winding layers over the 3D printed coil holder. In some embodiments, the AT coil device has 5 individual winding layers over the 3D printed coil holder. In some embodiments, the AT coil device has 6 individual winding layers over the 3D printed coil holder. In some embodiments, the AT coil device has 7 individual winding layers over the 3D printed coil holder. In some embodiments, the AT coil device has 8 individual winding layers over the 3D printed coil holder. In some embodiments, the AT coil device has 9 individual winding layers over the 3D printed coil holder.

In some embodiments, the 3D printed coil holder comprises a hole through the center for the accommodation of an optional core material. Without being bound by theory, it is believed that the core, comprising certain materials, increases the relative permeability of the AT coil device for different applications. With increased permeability the field focusing becomes better; there is a smaller focal spot size, and the coil depth will be reduced, which is particularly advantageous for small animal TMS coil applications. The other advantage is that the coil energy consumption will be reduced. In some embodiments, the core comprises a ferromagnetic material. In some embodiments, the core comprises iron. In some embodiments, the core comprises cobalt. In some embodiments, the core comprises nickel.

In some other embodiments, the winding wire comprises copper in general. In some embodiments, the winding wire is a litz wire. Without being bound by theory, an advantage of using litz wire is that it is flexible and easy to bend and the wires' cross-sections are fully utilized for current flows, e.g., at 5 kHz the skin depth for copper is about 1 mm, which is thicker than the diameter of the litz wires. In some embodiments, the winding wire is a multi-thread litz wire. In some embodiments, the diameter of the litz wire is in a range from about 0.2 mm (AWG 32) to about 0.4 mm (AWG 26). In some embodiments, the diameter of the litz wire is in a range from about 0.2 mm to about 0.3 mm. In some embodiments, the diameter of the litz wire is in a range from about 0.25 mm to about 0.3 mm. In some embodiments, the winding wire has 120 threads of 30 AWG insulated magnetic wires for flexible bending and high current operations. In some embodiments, the winding wire is not a copper bar or a copper strip having a larger cross-sectional area, e.g., about 10 mm×5 mm stripes or bars. In some embodiments, the AT coil device wires are insulated with epoxy resin. In some embodiments, the epoxy resin has a low value of viscosity before solidification to fill the space among the wires. The fabricated AT coil device's weight, optionally insulated in epoxy, is suitable for any coil support stand.

The tilting angle of the AT coil device can be in a range from about 0° to about 80°, relative to the horizontal plane, wherein the tilting angle is shown schematically in FIG. 1D. In some embodiments, the tilting angle of the AT coil device is in a range from about 10° to about 80°. In some embodiments, the tilting angle of the AT coil device is in a range from about 10° to about 50°. In some embodiments, the tilting angle of the AT coil device is in a range from about 20° to about 60°. In some embodiments, the tilting angle of the AT coil device is in a range from about 30° to about 70°. In some embodiments, the tilting angle of the AT coil device is in a range from about 40° to about 80°. In some embodiments, the tilting angle of the AT coil device is in a range from about 50° to about 70°. In some embodiments, the tilting angle of the AT coil device is in a range from about 60° to about 80°.

Each winding layer has an outer dimension and an inner dimension and is substantially circular, as shown in FIG. 1A, or substantially elliptical. In some embodiments, the width of the winding layer, or (outer diameter (OD) minus inner diameter (ID)), can be the same as, or different from, the width of each other winding layer, and is in a range from about 0.5 mm to about 5 mm. In some embodiments, the width of the winding layer is in a range from about 0.5 mm to about 3 mm. In some embodiments, the width of the winding layer is in a range from about 0.5 mm to about 2 mm. In some embodiments, the width of the winding layer is in a range from about 1 mm to about 2 mm. In some embodiments, the width of the winding layer is in a range from about 0.75 mm to about 1.5 mm. In some embodiments, the height of each winding layer can be the same as, or different from, the height of each other winding layer, and is in a range from about 0.5 mm to 2.5 cm high. In some embodiments, the height of each winding layer is in a range from about 0.5 mm to 5 mm. In some embodiments, the height of each winding layer is in a range from about 5 mm to 1 cm. In some embodiments, the height of each winding layer is in a range from about 1 cm to 1.5 cm. In some embodiments, the height of each winding layer is in a range from about 1.5 cm to 2 cm. In some embodiments, the height of each winding layer is in a range from about 2 cm to 2.5 cm.

The AT coil device is fabricated by wrapping wire over non-metal coil holders, wherein a coil holder outer diameter is substantially equal to the inner diameter of the winding layer, until the preferred width of the winding layer is achieved. An embodiment of a non-metal coil holder is shown in FIG. 1E, but it is not intended to limit same. It should be appreciated by the person skilled in the art that the coil holder outer diameter should have the same shape as that of the winding layer, for example, substantially circular or substantially elliptical. As shown in FIG. 1E, the non-metal coil holder comprises a coil holder inner diameter and a coil holder outer diameter, and together the coil holder inner and outer diameter have a toroidal shape. In some embodiments, the coil holder outer diameter (or the winding layer inner diameter) is in a range from about 1 cm to about 40 cm. In some embodiments, the coil holder outer diameter is in a range from about 1 cm to about 3 cm. In some embodiments, the coil holder outer diameter is in a range from about 1 cm to about 5 cm. In some embodiments, the coil holder outer diameter is in a range from about 5 cm to about 20 cm. In some embodiments, the coil holder outer diameter is in a range from about 5 cm to about 10 cm. In some embodiments, the coil holder outer diameter is in a range from about 6 cm to about 11 cm. In some embodiments, the coil holder outer diameter is in a range from about 7 cm to about 12 cm. In some embodiments, the coil holder outer diameter is in a range from about 8 cm to about 13 cm. In some embodiments, the coil holder outer diameter is in a range from about 9 cm to about 14 cm. In some embodiments, the coil holder outer diameter is in a range from about 10 cm to about 15 cm. In some embodiments, the coil holder outer diameter is in a range from about 11 cm to about 16 cm. In some embodiments, the coil holder outer diameter is in a range from about 12 cm to about 17 cm. In some embodiments, the coil holder outer diameter is in a range from about 13 cm to about 18 cm. In some embodiments, the coil holder outer diameter is in a range from about 14 cm to about 19 cm. In some embodiments, the coil holder outer diameter is in a range from about 15 cm to about 20 cm. The non-metal coil holder can be made of any polymeric material including, but not limited to, polylactic acid (PLA), acrylonitrile butadiene styrene (ABS), polyethylene terephthalate (PET), and thermoplastic polyurethane (TPU). Further referring to FIG. 1E, the non-metal coil holder further comprises winding layer separators, which have a wider diameter if circular, or width if non-circular, than the coil holder outer diameter. It should be appreciated that the distance between separators in the non-metal coil holder is substantially the same as the height of the winding layer. Although shown with two winding layers, it should be appreciated that the non-metal coil holders of FIG. 1E, or any equivalent embodiment thereof, can comprise upwards of nine winding layers. In some embodiments, a core material can be inserted into the hollow core defined by the coil holder inner diameter of the non-metal coil holder. A low-efficiency problem encountered by small diameter coils can be overcome using a core, e.g., a ferromagnetic core, which prevents the magnetic flux leakage out of the coil wall and enhances the field strength. Accordingly, in some embodiments, when the outer diameter of the winding layers is less than about 3 cm, a ferromagnetic core can be inserted into the hollow core defined by the coil holder inner diameter of the non-metal coil holder.

Other embodiments of the non-metal coil holder are envisioned, including a holder that permits the stacking of pre-manufactured, or modular, winding layers and separating layers in a sandwiched fashion (e.g., separating layer-(pre-manufactured winding layer-separating layer), wherein n=2-9; not shown) around a core, to resemble the structure of FIG. 1E. In yet another embodiment, the pre-manufactured winding layers can further comprise one or both separating layers such that upon stacking around a core, for example 2-9 stacking layers, the non-metal coil holder resembles the structure of FIG. 1E. In these embodiments, the pre-manufactured winding layers and separating layers comprise a hole in the center and each layer can be stacked and sandwiched around a core, for example a ferromagnetic core.

Accordingly, in a first aspect, an angled-tuned (AT) transcranial magnetic stimulation (TMS) coil device is described, said AT coil device comprising:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane of the non-metal coil holder.
   In some embodiments, the non-metal coil holder is monolithic. In some embodiments, the non-metal coil holder comprises a plurality of parts or modules that can be stacked together. In some embodiments, the separating layer(s) comprise the same material as the non-metal coil holder. In some embodiments, the separating layer(s) comprise different material than the non-metal coil holder, but is still non-metal. In some embodiments, the hollow core further comprises a core material that is different from the material of the non-metal coil holder. In some embodiments, the core comprises a material selected from the group consisting of ferromagnetic materials, iron, cobalt, and nickel. In some embodiments, the wire is litz wire. In some embodiments, the diameter of the litz wire is in a range from about 0.2 mm (AWG 32) to about 0.4 mm (AWG 26). In some embodiments, the winding wire is not a copper bar or a copper strip having a larger cross-sectional area, e.g., about 10 mm×5 mm stripes or bars. In some embodiments, the wire is insulated with epoxy resin. In some embodiments, the angle of the at least two winding layers is in a range from about 10° to about 80° relative to the horizontal plane of the non-metal coil holder. In some embodiments, the coil holder outer diameter is in a range from about 1 cm to about 40 cm. In some embodiments, two AT coil devices are arranged in a “V shape” relative to a horizontal plane. In some embodiments, two AT coil devices are arranged in an “A shape” relative to a horizontal plane. In some embodiments, four AT coil devices are arranged in a “V arrangement” containing two elliptical beam pairs (i.e., two AT coil devices are arranged in an “A shape” relative to a horizontal plane). In some embodiments, eight AT coil devices are arranged in two “V arrangements,” wherein each “V arrangement” contains two elliptical beam pairs (i.e., two AT coil devices are arranged in an “A shape” relative to a horizontal plane). In some embodiments, the angle of the two elliptical beam pairs in the “V arrangement” is in a range from about 30° to 70°. In some embodiments, the angle between each AT coil device in the elliptical beam pair is in a range from about 5° to 45°.

In a second aspect, a TMS system comprising at least one AT coil device of the first aspect is described. As described herein, TMS systems are known in the art, but the prior art TMS systems include conventional circular and figure-8 TMS coils, which occupy a substantial footprint. Therefore, it is challenging to accomplish more than two stimulation sites with the flexibility to move the coils around and reach the desired locations. Advantageously, the AT coil devices described herein can reduce the individual coil footprint and improve depth-spread characteristics in TMS systems.

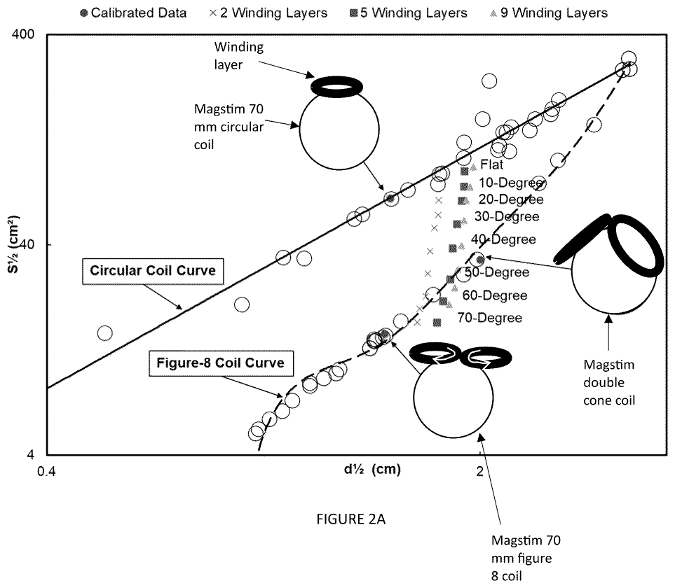

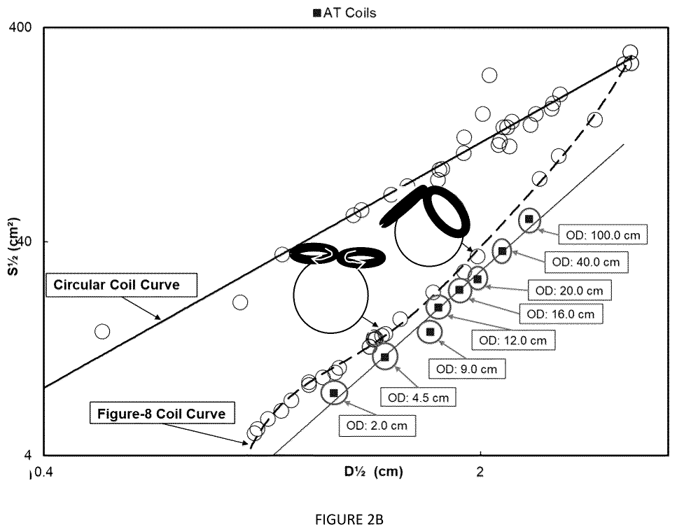

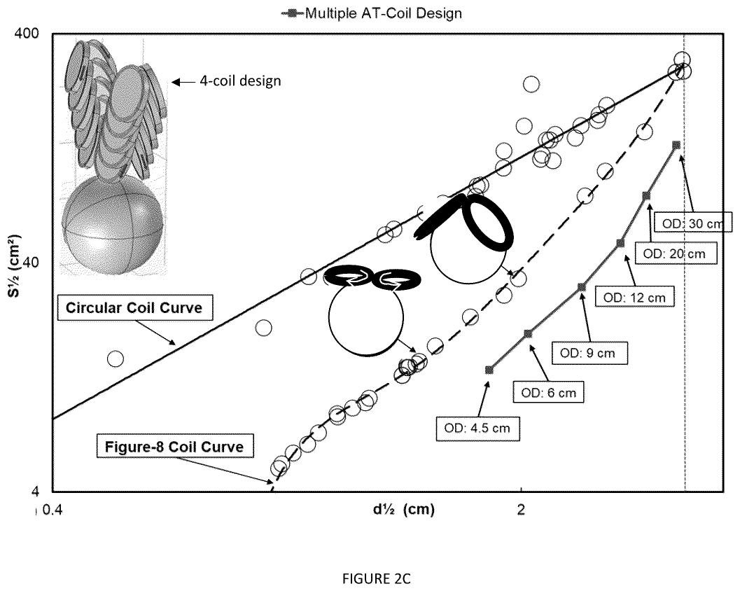

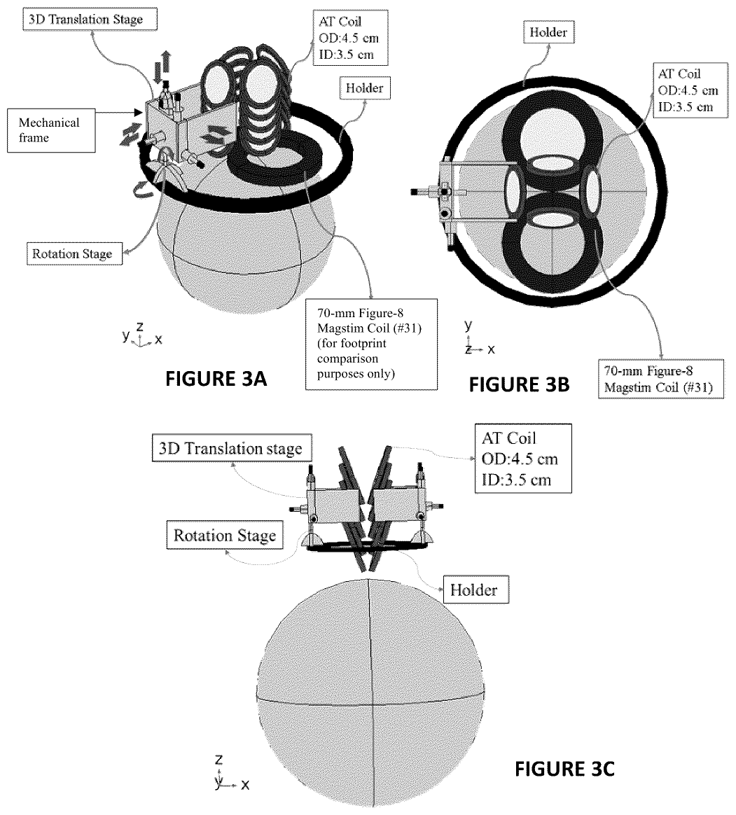

In some embodiments, the TMS system comprises 1, 2, 3, 4, 5, 6, 7, or 8 AT coil devices of the first aspect. In some embodiments, the TMS system comprises two or more AT coil devices and the TMS system is considered a multisite stimulator. It should be appreciated by the person skilled in the art that when there are two or more AT coil devices in the TMS system, each AT coil device can be the same as or different from (e.g., the number of winding layers, the tilting angle, the width of the winding layer, and the coil holder outer diameter) the other(s). In some embodiments, when there are two or more AT coil devices in the TMS system, they can be arranged around, or encircling, an imaginary axis. For example, in some embodiments, the two or more AT coil devices of the first aspect are arranged around the imaginary axis such that a “V shape” is formed (e.g., as shown in FIG. 3C). As defined herein, a “V shape” corresponds not only to the “V shape” formed by arranging two AT coil devices of the first aspect around the imaginary axis but also the conical shape formed by arranging three or more AT coil devices of the first aspect around the imaginary axis. In some embodiments, the angle of the longitudinal axis of each AT coil device, relative to the others, is substantially identical. For example, in some embodiments, the angle of the longitudinal axis of each AT coil device, relative to a X-Y axis, is 90°, while in some other embodiments, the angle of the longitudinal axis of each AT coil device, relative to a X-Y axis, is less than 90° or greater than 90° (see, e.g., the pairs in the 4-coil and 8-coil AT design). In some embodiments, the angle of the longitudinal axis of at least one AT coil device, relative to the others, is different than the others. In some embodiments, when paired, the angle between two AT coil devices can be in a range from about 5° to about 45°. In some embodiments, the two or more AT coil devices are arranged in the TMS system such that the apex of the “V shape” is positioned in proximity to a head (e.g., as shown in FIG. 3C). In some embodiments, the two or more AT coil devices are arranged in the TMS system such the apex of the “V shape” is positioned distally relative to the head (e.g., as shown in FIGS. 2C and 3A, also referred to as an “A shape”).

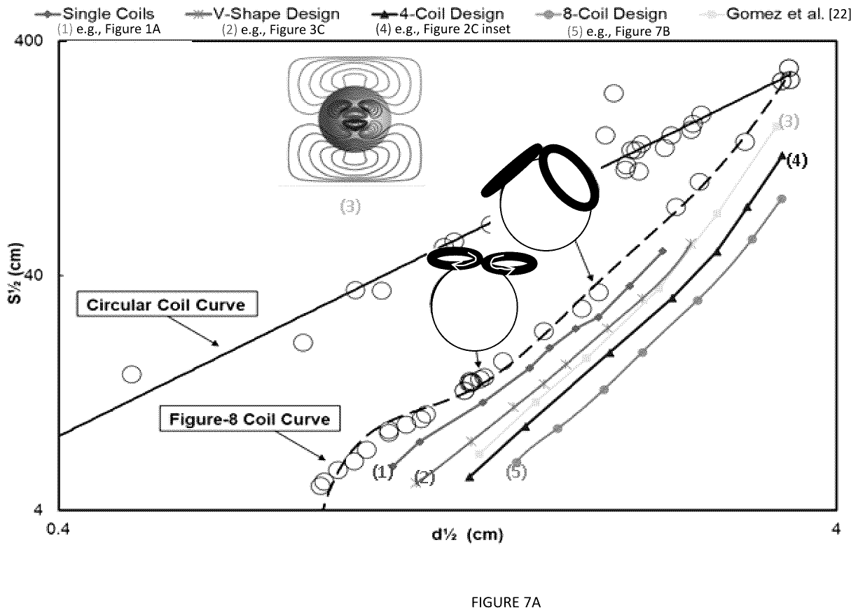

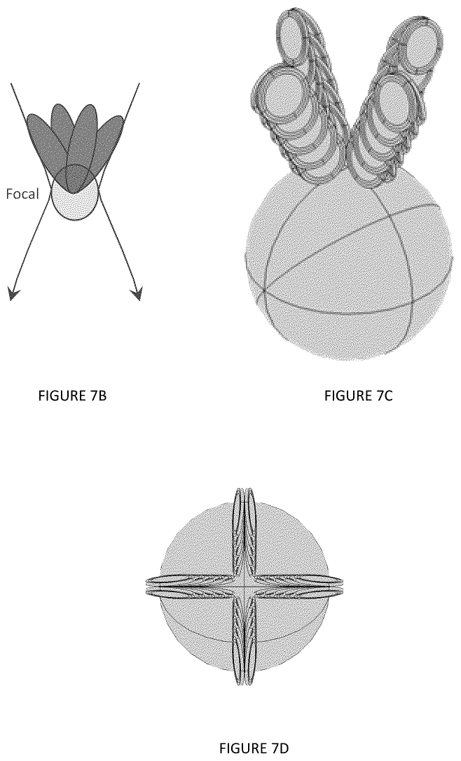

Notably, when two AT coil devices form an “A shape” pair (e.g., as shown in FIG. 3C), hereinafter an “elliptical-beam pair,” they produce an elliptical field distribution (see, e.g., one of the ellipses shown in FIG. 7B). With the ellipticity, the emitted elliptical beam shape is directional, which will help to produce field focusing. Since the generated fields must follow the emitting directions to the focused point first before they become diverged later, field focusing effect is produced and higher ellipticity helps better focusing. In some embodiments, the angle between two AT coil devices in the elliptical beam pair can be in a range from about 5° to about 45°. Adding another AT coil to form a pair and having two AT coil pairs with opposite polarities can create a symmetric structure and produce a more elliptical field distribution. Two or four of these elliptical beam pairs form a composite coil and together they produce a field focusing effect and have less divergent emitting field pattern. Accordingly, in some embodiments, two elliptical beam pairs are arranged to form the composite coil shown in FIG. 2C (also referred to as a 4-coil design). In this embodiment, the 4-coil design comprises two separate elliptical beam pairs are positioned in a “V arrangement,” wherein the “V arrangement” angle is in a range from about 30° to about 70°. Without being bound by theory, it is believed that the “V arrangement” angle contributes to the final optimized depth-spread performance. The two “A-shape” AT coil pairs are providing two elliptical beams and the two beams join at the TMS system's focal spot. As the “V arrangement” angle increase, the depth becomes shallower. As the “V arrangement” angle is decreased, the spread increases. Accordingly, there is an optimized “V arrangement” angle to obtain the best depth-spread performance. In some embodiments, four elliptical beam pairs are arranged to form the composite coil shown in FIGS. 7C and 7D (also referred to as an 8-coil design). In this embodiment, the 8-coil design comprises four separate elliptical beam pairs, and two separate “V arrangement,” having the same apex or focal point. Similarly, the “V arrangement” angle is in a range from about 30° to about 70°.

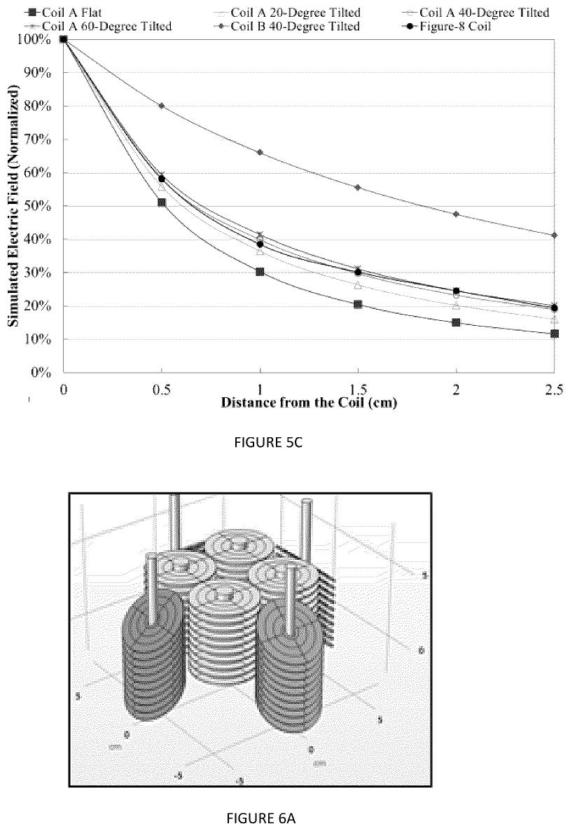

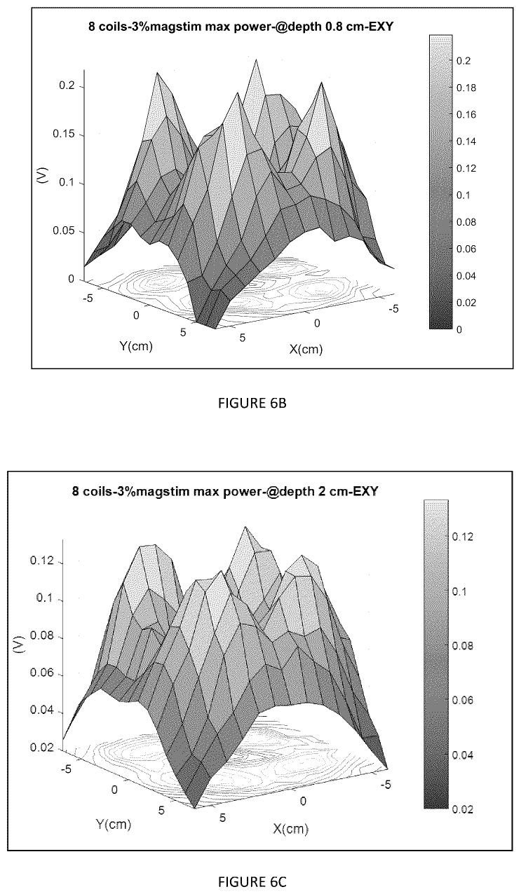

As introduced hereinabove, the tilting angle of the AT coil devices can be 0°. In some embodiments, the TMS system is a multisite stimulator comprising three or more AT coil devices of the first aspect, wherein the tilting angle of at least one of the AT coil devices is 0°, and wherein the 0° AT coil device is positioned between at least two AT coil devices having tilting angles between 10° and 80°, as described herein. For example, FIG. 6A shows an example of a 4-site multi-focused stimulator made of 8 small coils, its implementation, and measured results. The small coils can be flat or angled at different locations. In general, flat coils are appropriate for the middle and accomplish interference cancelation, while the angled AT coils can surround the flat coilsto generate sharp stimulation peaks. Accordingly, in one embodiment, the TMS system comprises eight AT coil devices, wherein four of the AT coil devices have a tilting angle of 0° and four of the AT coil devices have a tilting angle between 10° and 80°, wherein the 0° AT coil devices are arranged around, and closer to, the imaginary axis and the angled AT coil devices are arranged around the 0° AT coil devices. In another embodiment, the TMS system comprises five AT coil devices, wherein one of the AT coil devices has a tilting angle of 0° and four of the AT coil devices have a tilting angle between 10° and 80°, wherein the 0° AT coil devices are arranged at the imaginary axis and the angled AT coil devices are arranged around the 0° AT coil device. In yet another embodiment, the TMS system comprises three AT coil devices, wherein one of the AT coil devices has a tilting angle of 0° and two of the AT coil devices have a tilting angle between 10° and 80°, wherein the 0° AT coil device is arranged at the imaginary axis and the angled AT coil devices are arranged around the 0° AT coil device.

It should be appreciated that the AT coil devices of the first aspect can be attached to a mechanical frame for adjustment purposes, for example following the instructions of a computer program product, as understood by the person skilled in the art. For example, referring to FIGS. 3A-3C, each AT coil device can be attached to a dedicated mechanical frame for adjustment relative to a head. In some embodiments, the mechanical frame includes a 3D translation stage, a rotation stage, or both a 3D translation stage and a rotation stage. Although not shown, for the sake of clarity, the four AT coil devices of the first aspect, as shown in FIG. 3A, can each have their own dedicated mechanical frame. In some embodiments, the mechanical frame can be used to adjust the angle of the longitudinal axis of each AT coil device in the TMS system. In some embodiments, the mechanical frame can be used to adjust the position, along the X-Y plane, of each AT coil device in the TMS system. In some embodiments, the mechanical frame can be used to adjust the position, along the Z plane, of each AT coil device in the TMS system. In some embodiments, the mechanical frame can be used to rotate the apex of the “V shape” or “V arrangement” to be in proximity to the head. In some embodiments, the mechanical frame can be used to rotate the apex of the “V shape” so that it is distally arranged relative to the head. In should be appreciated however, that more than one AT coil device can be associated, or attached to, one mechanical frame, as understood by the person skilled in the art. In some embodiments, the mechanical frames are made of non-metal material including, but not limited to, plastic or ceramic materials. In some embodiments, the mechanical frames are attached to a holding means.

Accordingly, in an embodiment of the second aspect, a TMS system is described, said TMS system comprising:
a mechanical frame; and
at least one AT coil device attached to the mechanical frame for adjustment of the at least one AT coil device in the TMS system,
wherein the at least one AT coil device comprises:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane of the non-metal coil holder.
   In some embodiments, the non-metal coil holder is monolithic. In some embodiments, the non-metal coil holder comprises a plurality of parts or modules that can be stacked together. In some embodiments, the separating layer(s) comprise the same material as the non-metal coil holder. In some embodiments, the separating layer(s) comprise different material than the non-metal coil holder, but is still non-metal. In some embodiments, the hollow core further comprises a core material that is different from the material of the non-metal coil holder. In some embodiments, the core comprises a material selected from the group consisting of ferromagnetic materials, iron, cobalt, and nickel. In some embodiments, the mechanical frame is a non-metal frame. In some embodiments, the wire is litz wire. In some embodiments, the diameter of the litz wire is in a range from about 0.2 mm (AWG 32) to about 0.4 mm (AWG 26). In some embodiments, the winding wire is not a copper bar or a copper strip having a larger cross-sectional area, e.g., about 10 mm×5 mm stripes or bars. In some embodiments, the wire is insulated with epoxy resin. In some embodiments, the angle of the at least two winding layers is in a range from about 10° to about 80° relative to the horizontal plane of the non-metal coil holder. In some embodiments, the coil holder outer diameter is in a range from about 1 cm to about 40 cm. In some embodiments, two AT coil devices are arranged in a “V shape” relative to a horizontal plane. In some embodiments, two AT coil devices are arranged in an “A shape” relative to a horizontal plane. In some embodiments, four AT coil devices are arranged in a “V arrangement” containing two elliptical beam pairs (i.e., two AT coil devices are arranged in an “A shape” relative to a horizontal plane). In some embodiments, eight AT coil devices are arranged in two “V arrangements,” wherein each “V arrangement” contains two elliptical beam pairs (i.e., two AT coil devices are arranged in an “A shape” relative to a horizontal plane). In some embodiments, the angle of the two elliptical beam pairs in the “V arrangement” is in a range from about 30° to 70°. In some embodiments, the angle between each AT coil device in the elliptical beam pair is in a range from about 5° to 45°. In some embodiments, the TMS system does not comprise more than eight elliptical beams.

In some embodiments of the second aspect, a TMS system is described, said TMS system comprising:
a mechanical frame; and
at least two AT coil devices attached to their own dedicated mechanical frame for adjustment of the at least two AT coil devices in the TMS system, wherein the at least two AT coil devices are arranged in a “V shape” or an “A shape,” relative to a horizontal plane,
wherein the at least one AT coil device comprises:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane.
   In some embodiments, the non-metal coil holder is monolithic. In some embodiments, the non-metal coil holder comprises a plurality of parts or modules that can be stacked together. In some embodiments, the separating layer(s) comprise the same material as the non-metal coil holder. In some embodiments, the separating layer(s) comprise different material than the non-metal coil holder, but is still non-metal. In some embodiments, the hollow core further comprises a core material that is different from the material of the non-metal coil holder. In some embodiments, the core comprises a material selected from the group consisting of ferromagnetic materials, iron, cobalt, and nickel. In some embodiments, the mechanical frame is a non-metal frame. In some embodiments, the wire is litz wire. In some embodiments, the diameter of the litz wire is in a range from about 0.2 mm (AWG 32) to about 0.4 mm (AWG 26). In some embodiments, the winding wire is not a copper bar or a copper strip having a larger cross-sectional area, e.g., about 10 mm×5 mm stripes or bars. In some embodiments, the wire is insulated with epoxy resin. In some embodiments, the angle of the at least two winding layers is in a range from about 10° to about 80° relative to the horizontal plane of the non-metal coil holder. In some embodiments, the coil holder outer diameter is in a range from about 1 cm to about 40 cm. In some embodiments, the TMS system comprises 2, 3, 4, 5, 6, 7, or 8 AT coil devices, wherein the AT coil devices are the same as or different from one another. In some embodiments, four AT coil devices are arranged in a “V arrangement” containing two elliptical beam pairs (i.e., two AT coil devices are arranged in an “A shape” relative to a horizontal plane). In some embodiments, eight AT coil devices are arranged in two “V arrangements,” wherein each “V arrangement” contains two elliptical beam pairs (i.e., two AT coil devices are arranged in an “A shape” relative to a horizontal plane). In some embodiments, the angle of the two elliptical beam pairs in the “V arrangement” is in a range from about 30° to 70°. In some embodiments, the angle between each AT coil device in the elliptical beam pair is in a range from about 5° to 45°. In some embodiments, the TMS system comprises at least one AT coil device having a tilting angle of 0° and at least two AT coil devices having a tilting angle of 10° to 80°. In some embodiments, the TMS system does not comprise more than eight elliptical beams.

The AT coil design described herein improves the depth-spread performance of individual coils with a significantly smaller footprint than existing coils. For composite structures, using the AT coil design described herein as basic building blocks simplifies the design and manufacturing process and helps accomplish a leading depth-spread performance. In addition, the footprint of the AT coil device is intrinsically small, making them suitable for multisite stimulations of inter and intra-hemispheric brain regions with an improved spread and less electric field divergence. Since few brain functions are operated by isolated single brain regions but rather by coordinated networks involving multiple brain regions, simultaneous or sequential multisite stimulation may provide tools for mechanistic studies of brain functions and the treatment of neuropsychiatric disorders.

### Computer Program Products

The present subject matter described herein may be a system, a method, and/or a computer program product. In some embodiments, the computer program product may include a computer readable storage medium (or media) having computer readable program instructions thereon for causing a processor to carry out aspects of the present subject matter.

In some embodiments, the computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

In some embodiments, the computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In some embodiments, the computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In some embodiments, the computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

### Example 1

### Experimental

FIG. 1D shows an embodiment of the manufactured coil for the experimental measurements described herein. A Magstim 200 (Magstim Co Ltd, Whitland, UK.) stimulator was used to drive these coils with the power set at 30% during the measurements, producing about 480V on the discharging capacitor. Using a 30% power rating to conduct the field distribution measurements is necessary since it obtains reliable measurement results while not pushing the instrument to its operation lifetime limit due to the high number of trials. The coil's electric field distribution was measured in a 5 mm step using calibrated high-spatial-resolution vector-field probes [19]. The total field strength, |E|=(Eₓ²+E_{y}²+E_{z}²)^{1/2}, is obtained from measuring the x, y, and z-direction field components at each step within the X-Y planes. The obtained values were normalized for easy comparison between experimental and simulation results.

FIG. 1A illustrates an embodiment of the TMS coil design described herein. The coil has a fixed winding width (the difference between the outer diameter (OD) and inner diameter (ID) of the coil), for example, about 1 cm. In the simulations, a variety of the windings' inner and outer diameters were used. In addition, the coil stacking number included 2, 5 (FIG. 1A), and 9 (FIG. 1D) stacks along the central axis (Z-axis) with a tilting angle of up to 70 degrees with a total height of 12.0 cm, 21.8 cm, and 34.8 cm, respectively. The layers were connected in series; the current excitation in all coils is a sinusoidal wave with a frequency of 5 kHz. In some embodiments, a 0.5 cm layer of insulation can be placed between the coil and the spherical head surface.

FIG. 1B illustrates the definition of stimulation depth and spread in the head model introduced by the half-value depth (d_{1/2}), half-value spread (S_{1/2}), and half-value volume (V_{1/2}) described in Deng et al. [20] The purpose for defining these specific metrics and considering the half value of Eₘₐₓ[21], is to ensure the independence of the coil's performance regarding the electric field's distribution from the absolute Eₘₐₓ value since all the obtained data are normalized to Eₘₐₓ. The absolute intensity of the electric field can be modulated through the TMS stimulator circuit and has no significance in the spatial distribution hence being excluded from these analyses.

To compare these simulations to previous studies, three coils were selected (70 mm circular (#4), 70 mm figure-8 (#31), and double cone (#37) Magstim coil) and the same coil parameters used in Deng et al. were used [20]. The half-value depth and spread of the three coils were analyzed. For all three cases, the simulated depth-spread results are within 1% of previously reported results (see FIG. 2A).

FIG. 1C demonstrates the definition of footprint, characterized as the tangential surface area that the coil occupies in the projected surface plane. For example, the 70-mm figure-8 Magstim coil has a lateral surface area of 120 cm², making it difficult to operate two coils on the human head simultaneously. For the AT coils, the footprint is the coil's projection area on the head model surface, dependent on the tilting angle. For example, the AT coil with an outer diameter of 4.5 cm has a footprint range of about 5.5 cm² to 15.9 cm² for 70-degree tilted coils to flat coils. Thus, the footprint for the 70-degree tilted coil is approximately 95% less than the figure-8 coil. The smaller footprint of the coils allows for the use of more coils over the subject's head for multisite stimulation.

FIG. 2A-2C are depth-spread plot to form the best-fit curves for circular coils (solid line) and figure-8 coils (dashed line). Two curves were used as references to illustrate how different coil design parameters, such as tilting angle, number of winding layers, coil location, rotation angle, and outer diameter size, can affect the performance in terms of locations in the S_{1/2} vs. d_{1/2} plot. The maximum points for S_{1/2} and d_{1/2} in the plot have been defined based on exposing the spherical head model to the induced field from a symmetric spherical coil covering the whole head [20]. FIG. 2A illustrates the AT coils' performance with an inner diameter of 8 cm and an outer diameter of 9 cm with tilting angles ranging from 0 to 70 degrees with 10-degree steps and stacking numbers of 2, 5, and 9. When the tilting angle is increased from 0 degrees (flat circular coil stack) to 70 degrees, the spread is significantly reduced with a slight reduction in the depth. The reduction in the depth may be due to the tilting effect since part of the coil is pulling away from the head model. It can be seen that the coils' depth-spread performance surpasses the figure-8 coil curve when the tilting angle reaches about 50-60 degrees and beyond. Without being bound by theory, this improvement appears to show that the primary effect of the angle-tuning is to reduce the field spread. On the other hand, when the coil stacking number increases, the depth performance improves. This effect seems to be saturated from 5 to 9 coils. Both reducing the spread or increasing the depth helps to reduce the divergence of the emitting field and make it more elliptical than spherical. To check one numerical example, the depth-spread of a five-winding-layer, 70-degree tilted coil was compared with the 70-mm figure-8 Magstim coil (#31). They have a similar spread, but the AT coil has a 20% deeper half-depth and a smaller footprint by about 82%, as the figure-8 coil has a footprint of about 120 cm² and the AT coil has a footprint of 22 cm².

To obtain the tradeoff curve, the coils' outer diameter were varied between 2 cm and 100 cm while keeping the winding width constant at 1 cm. The tilting angle was fixed at 70 degrees, and the rotation angle was 20 degrees, with the lower edge of the coil aligned with the head model's central axis. FIG. 2B shows the results of the depth-spread performance of these coils as a function of coil diameter. The AT coils have a smaller footprint and demonstrate a better tradeoff curve than conventional figure-8 coils. For example, an AT coil with an inner and outer diameter of 3.5 cm and 4.5 cm, respectively, establishes the same d_{1/2} as the figure-8 coil (coil #31) with a 10% smaller spread. When the coil's inner and outer diameters reach 8.0 cm and 9.0 cm, respectively, the half-value depth experienced a 20% increase compared to the figure-8 coil (coil #31) with the same spread (S_{1/2}). In addition, if compared with the double cone coil, the AT coil with an outer diameter of 20 cm demonstrates the same half-value depth with a 20% smaller spread. This is a considerable improvement compared to the existing figure-8 coils, given that the AT coil occupies a significantly smaller footprint.

AT coils can also be used as fundamental building blocks for coils with more complex structures and better depth-spread performance. A single AT coil is not symmetric, and it can occupy more V_{1/2} in the head model. Adding another AT coil to form a pair and having two AT coil pairs with opposite polarities can create a symmetric structure and produce a more elliptical field distribution. By adjusting various angles among these pairs, we can further optimize the depth-spread performance and obtain an even better depth-spread tradeoff curve. FIG. 2C shows an implemented example using this concept. The AT coils' arrangement in the structure is inserted in the top left corner with red arrows demonstrating each coil's current direction. In this design, two 80-degree tilted coils with an angle of 20 degrees between them form a pair; the coil design includes two of these pairs with opposite polarities and an internal angle of 60 degrees between them, and the coil ID changes from 3.5 to 29 cm with a 1 cm winding width. This result indicates a further improvement of the S_{1/2} and d_{1/2} and confirms AT coils' role as building blocks for complex coil structures. Compared with the commercial 70-mm figure-8 coil (coil #31), the 3.5 cm inner diameter 4-AT-coil module demonstrates a 10% smaller spread and a 30% larger din. The design with a 5 cm inner diameter 4-AT-coil module demonstrates a 15% larger spread and a 50% larger d_{1/2} than the corresponding figure-8 coil. The same coil has the same d_{1/2} with a 40% smaller spread than the double cone coil. The coils demonstrate a better depth-spread performance than the existing coils.

The AT coils have demonstrated a significant potential for multisite brain stimulation. FIGS. 3A-3C illustrates an embodiment of an apparatus and the implementation of multisite stimulation using said apparatus. In FIG. 3A, four 4.5-cm AT coil stacks in a ring structure are shown, wherein one is illustrated as placed on a mechanical frame (non-metal holder). In order to simplify the schematic, only one mechanical frame is shown in FIGS. 3A and 3B, accommodating one stacked AT coil. In other words, it should be appreciated that, in practice, each stacked AT coil in FIGS. 3A and 3B has its own mechanical frame. Each coil's relative 3-D location is adjustable and trackable through the translation and rotation stages located on the mechanical frame. These stages can be motorized to track all the relative locations. The designed system is convenient to adapt to individual head differences. It has an enhanced depth-spread tradeoff and a small contact area. The 4.5-cm AT coils were used for this design since they establish a smaller spread with a smaller footprint in comparison to conventional coils; the conventional 70-mm figure-8 coil (#31) (shown for comparison to demonstrate the smaller footprint of the AT coil; not part of the apparatus) and the 4.5-cm AT coil have the same half-depth with a 10% smaller spread and 95% smaller footprint for the single AT coil, as shown in FIG. 3B. The smaller footprint and contact surface of the AT coils, in addition to the enhanced depth-spread tradeoff, create flexibility with the coil location and movement relative to the head and the number of coils on the scalp in comparison to conventional dual-coil systems. FIG. 3C demonstrates the system's capability to generate two stimulation points as close to each other as 1 cm. The mechanical frame should not be counted toward the footprint of the apparatus as it is a supporting structure for performing multisite stimulation.

### Experimental Verification

To verify the simulated data, a total of 8 coil prototypes were fabricated with two different dimensions, two different winding layers, and different tilting angles. The electric field distributions were measured for each of the AT coils and the commercial 70 mm figure-8 Magstim coil using calibrated high-spatial-resolution vector-field probes. The first coil, “Coil-A,” has an inner and outer diameter of 1 cm and 3 cm, respectively, with nine winding layers. The tilting angle ranges from 0 to 60 degrees with a step of 10 degrees. The second coil, “Coil-B,” has an inner diameter of 3 cm and an outer diameter of 9 cm with six winding layers with a tilting angle of 40 degrees. For comparison, both COMSOL simulations and experimental measurements of the electric field decay rate and stimulation hot spot area were conducted.

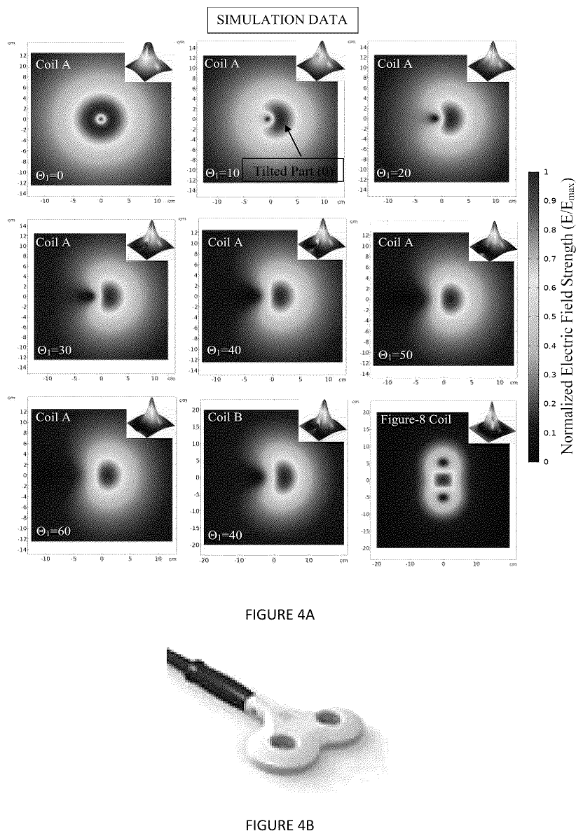

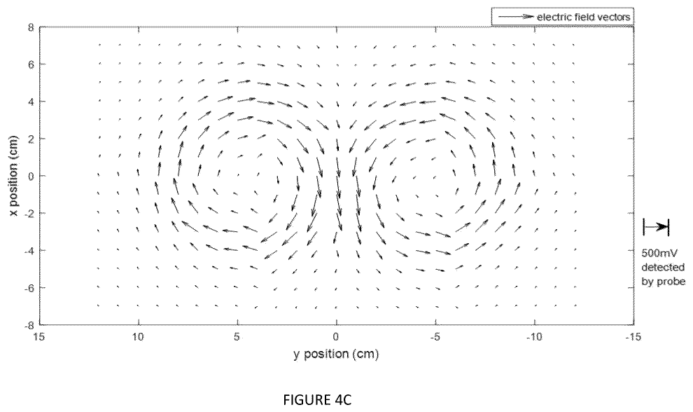

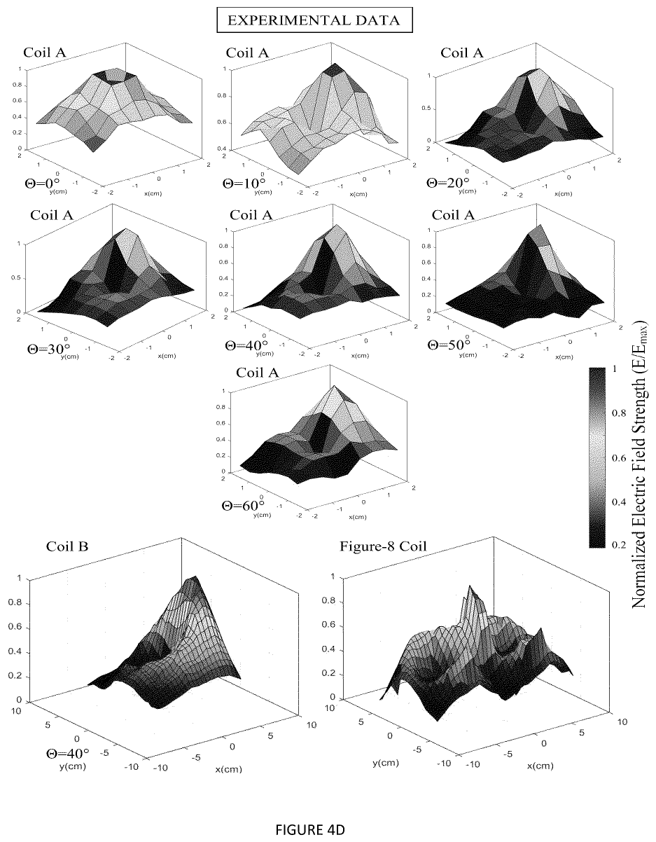

FIG. 4A shows FEM simulation and FIG. 4D shows experimental measurement results of electric field distributions at 1.5 cm away from the surface of these coils in air. The results indicate that, for the AT coils, the location of the maximum electric field is almost the same, close to the tilted edge of the coil, while for figure-8 coils, the maximum electric field is in the center of the coil. Experimental implementation and focal stimulation verifications based on electric field probe measurements. The high spatial resolution vector field probe employed is a miniaturized Rogowski coil. It has been used to characterize a standard Magstim D-70 flat coil as shown in FIG. 4B and obtain 2-D vector field plot of the D-70 figure-8 coil emission pattern as shown in FIG. 4.

Although it is difficult to measure the S_{1/2} directly, one way to represent the spread properly is to check the size of the hot spot as defined in the following procedure. The coils were scanned and the maximum electric field strength at a fixed distance away from the coils was obtained. At each distance, hot spot size is defined by measuring the area with the electric field strength above a selected percentage of that measured maximum strength. For example, at the distance of 1.5 cm away from the coils, the hot spot size was defined as the areas with an electric field intensity of more than 90% of the measured maximum electric field strength. Greater than 90% was chosen to avoid the need to scan a larger area for a smaller percentage without losing fairness and accuracy in the evaluations.

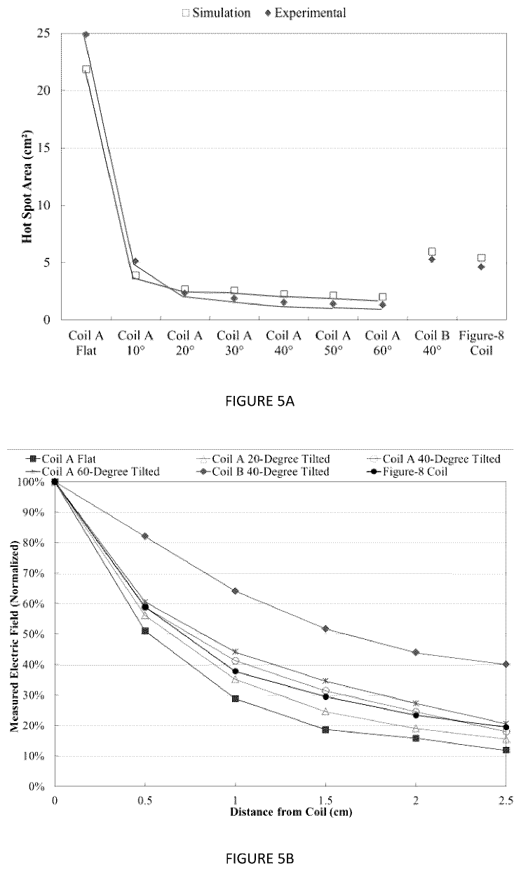

As shown in FIG. 5A, tilting the wrapping angle of the coil has a significant effect on decreasing the focal spot size. At a 10-degree tilted angle, the spot size drops 80% from that of the flat coil. The spot size further reduced an additional 70% from around 5.0 cm² for the 10-degree coil to about 1.5 cm² for the 60-degree coil. The focal spot size of the entire coil-A series is smaller than that of the figure-8 coil. On the other hand, the 40-degree tilted coil-B has a slightly bigger focal spot area than the studied figure-8 coil, which can be overcome if higher tilting angles are used. The argument can be proved by using the coil-A series as an example. The hot spot size reduced 15% from a 40-degree coil to a 60-degree coil. If the same effect is applied, the hot spot size of the B coil becomes smaller than that of the figure-8 coil.

The electric field intensity decay rates based on the experimental data and simulations are shown in FIGS. 5B and 5C, respectively. First the measured data was normalized for an accurate comparison. As shown in these plots, the decay rates gain improvements by increasing the tilting angle. At a depth of 2.5 cm from the coil, the decayed remaining value of the flat coil is less than 15.0% for both experimental and simulation data. In contrast, for the 60-degree tilted coil, the value increases to 25.0%, indicating an improvement of the decay rate. It can also be observed that the 40-degree tilted coil B has a much slower decay rate than the figure-8 and all other coils. The depth-spread performance of the AT coils is summarized in Table 1. The hot spot area of A coils is smaller than the figure-8 coil, while the hot spot area of the B coil is comparable to the figure-8 coil. Nevertheless, its decay rate is more than twice slower than the figure-8 coil. More than 40% of field strength remained at 2.5 cm distance for the B coil compared with the figure-8 coil with less than 20% left. Additionally, the simulation data in FIG. 2 shows the improvement in performance for higher tilting angles compared to the 40-degree tilted coil.

TABLE 1
The hot spot area and the induced electric field intensity decay
rates from both experimental measurements and FEM simulations.

| | | Hot Spot Area (cm²) | | Remaining % at 2.5 cm | |
|---|---|---|---|---|---|
| Coil | Tilting | Measured | Simulated | Measured | Simulated |
| Type | Angle | value | value | value | value |
| Coil A | Flat | 24.88 | 21.89 | 11.8 | 11.6 |
| | 10 | 5.13 | 3.9 | 13.6 | 14.1 |
| | 20 | 2.34 | 2.7 | 15.6 | 16.1 |
| | 30 | 1.85 | 2.57 | 16.7 | 17.9 |
| | 40 | 1.52 | 2.28 | 17.9 | 18.9 |
| | 50 | 1.41 | 2.13 | 19.0 | 19.6 |
| | 60 | 1.3 | 2.01 | 20.5 | 20.1 |
| Coil B | 40 | 5.28 | 5.98 | 40 | 41.1 |

| | | | | |
|---|---|---|---|---|
| FIG.-8 | 4.61 | 5.44 | 19.4 | 19.3 |

One notable fact is that for the 50- and 60-degree tilted angles, the small diameter A coils can accomplish better decay rates than that of the much larger diameter figure-8 coil. This performance shows that the design can produce elevated electric field intensity in deeper brain regions due to the field redistribution, or more precisely, focusing effect through angle tuning.

Advantageously, it is possible to simultaneously stimulate multiple sites at close distances using an embodiment of the apparatus described herein. FIG. 6A shows an example of a 4-site multi-focused stimulator made of 8 small coils, its implementation, and measured results. The small coils can be flat or angled at different locations. In general, flat coil is good to be in the middle and accomplish interference cancelation and angled ones can be put at the side to generate sharp stimulation peaks. Doing this way helps to accomplish good peak and valley distinction. As shown in FIG. 6C, the measured stimulation pattern indeed has distinct peaks and valleys 2 cm away from the coil. With the same spirit, we can develop 1- to 3-site stimulation patterns by simply removing 3 to 1 of the 4-angled coil(s), respectively, and accomplish 1-3 distinct stimulation peaks. The distance between these peaks can be adjustable by reducing or adding flat coils in-between angled stimulation coils.

### Discussion

Improving depth-spread performance by reducing field divergence through creating a more elliptical emitted field distribution from the coil. To accomplish that, instead of enriching the Fourier components along the planarized (x-y) directions, which requires different arrays to occupy large brain surface areas, the radial (z) direction was used by using tilted coil angles and stacking coil numbers to reduce the divergence of the emitted near field without occupying large head surface areas.

The coil design described herein has the advantage of occupying a much smaller contact surface during the stimulation due to its vertical stacking. For multisite brain stimulation, only one power supply circuit unit is required for the AT coils. Since all the elements are the same with an equal inductance, the combined inductance can easily be adjusted with parallel and serial connections to further improve depth-spread performance and multisite stimulation. The approach of using a uniform building block to construct a composite coil structure provides other benefits. First, mass production of identical small units can help to reduce cost and increase quality. Second, it provides flexibility in designing and implementing a novel generation of TMS tools by merely adjusting the relative geometric locations of these identical building block coils. The single AT coils used in the composite structure can also be adjusted with the tilting angle and the stacking number to match the required stimulation results. Third, the AT coils' simple design allows for easier replacement of possible defective elements in the multi-coil apparatus versus repairing or replacing the whole unit like other reported complicated multisite stimulation structures. Noticeably, compared with the reported or existing complex coil structures designed for deep brain stimulation, our simple four-uniform-AT-coil design has better depth-spread performance, as shown in FIG. 2C. This coil design provides a promising new generation of future high depth-spread performance and multisite TMS tools.

With higher inductance (about 65 μH) than figure-8 coils (about 16 μH), the coil design requires the same current to induce equal electric field strength in the brain but requires higher power consumption. It has been shown that increasing the tilting angle has a negative effect on the Energy requirements while causing a significant improvement in the electric field distribution focality and the footprint. In addition, increasing the winding layers has an adverse effect on energy consumption; in fact increasing the winding layers beyond a specific number is ineffective in the performance of the coil. For the complex coil system introduced in our work (FIG. 2C), the depth performance is significantly better than the commercial figure-8 coils, while the required energy is higher.

While many of the coils described herein have shown higher energy requirements in comparison to figure-8 coils, they have a significantly better depth performance to reach deeper regions of the brain. In addition, the energy consumption of all these AT coils are still in the range that can be driven by mainstream commercial TMS power supplies.

The coil design wires are flexible, enhancing the sound created by the Lorentz force. In some embodiments, to minimize the sound created by the Lorentz force, the flexible AT coil wires are insulated with epoxy resin. In some embodiments, the epoxy resin has a low value of viscosity before solidification to fill the space among the wires. It should be appreciated by the person skilled in the art that other containment and attenuation methods can further reduce the noise generation of the proposed coils.

To study the induced maximum electric field intensity (Eₘₐₓ), an arbitrary current of 3 kA, an acceptable value for all the commercial TMS stimulators, was applied to the three different coils; 70 mm Figure-8 (#31), AT OD-9 cm 70°, and small figure-8. The induced Eₘₐₓ at different radial distances from the head surface was analyzed. Although not shown, the data indicates that the AT coils described herein demonstrate a higher field strength at deeper brain regions. In contrast, conventional 70 mm figure-8 coil shows higher intensities at distances closer to the head surface. This data further verifies the performance of AT coils for stimulation of deeper brain regions.

Although not shown, the AT coil described herein can induce unilateral movements in anesthetized mice and rats. The voltage of the coil to reach the motor threshold was lower than 1 kV. Advantageously, experiments show that only the motor cortex region corresponding to the right-side hindlimb is stimulated, which not only validates that the probe measurements can be a sufficient method to determine and calibrate coil performance but also shows that the activation spot occupies a very small area in the brain in the millimeter range.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

1. Costello A 2011 Repetitive transcranial magnetic stimulation (rTMS) systems—class II special controls guidance for industry and FDA staff.
2. Caccomo S 2018 FDA permits marketing of transcranial magnetic stimulation for treatment of obsessive compulsive disorder FDA News Release.
3. Anand S and Hotson J 2002 Transcranial magnetic stimulation: neurophysiological applications and safety Brain Cogn. 50 366-86
4. Barker A T and Shields K 2017 Transcranial magnetic stimulation: basic principles and clinical applications in migraine Headache J. Head. Face Pain 57 517-24.
5. Lisanby S H, Luber B, Perera T and Sackeim H A 2000 Transcranial magnetic stimulation: applications in basic neuroscience and neuropsychophamtacology Int. Neuropsychopharmacol. 3 259-73.
6. Bullmore E and Spores O 2009 Complex brain networks: graph theoretical analysis of structural and functional systems Nat. Rev. Neurosci, 10 186-98
7. Hutchison R M, Womelsdorf T, Gati J S, Everting S and Menon R S 2013 Resting-state networks show dynamic functional connectivity in awake humans and anesthetized macaques: dynamic functional connectivity Hum. Brain Mapp. 34 2154-77
8. Koch G and Rothwell J C 2009 TMS investigations into the task-dependent functional interplay between human posterior parietal and motor cortex Behav. Brain Res. 202 147-52
9. Rothwell J C 2011 Using transcranial magnetic stimulation methods to probe connectivity between motor areas of the brain Hum. May, Sci. 30 906-15
10. Koganermaru S, Mima T, Nakatsuka M, Uek, Y, Fukuyama H and Domen K 2009 Human motor associative plasticity induced by paired bihemispheric stimulation: paired bihemispheric stimulation induces motor plasticity J. Physiol. 587 4629-44
11. Buch E R. Johnen V M, Nelissen N, O'Shea J and Rushworth M F S 2011 Noninvasive associative plasticity induction in a corticocortical pathway of the human brain J. Neurosci. 31 17669-79
12. Lu M-K, Tsai C H and Ziemann U 2012 Cerebellum to motor cortex paired associative stimulation induces bidirectional STDP-like plasticity in human motor cortex Front. Hum. Neurosci. 6 260
13. Takeuchi N, Tada T, Toshima M and Ikoma K 2010 Correlation of motor function with transcallosal and intracortical inhibition after stroke J. Rehabil. Med. 42 962-6
14. Schmitt A, Hasan A, Gruber O and Falkai P 2011 Schizophrenia as a disorder of disconnectivity Eur. Arch. Psychiatry Clin. Neurosci. 261 150-4
15. Farzan, F, Vernet M, Shaft M M D, Rotenberg A, Daskalakis Z J and Pascual-Leone A 2016 Characterizing and modulating brain circuitry through transcranial magnetic stimulation combined with electroencephalography Front. Neural Circuits 10 73
16, Cash R F H, Cocchi L, Anderson R, Rogachov A, Kucyi A, Barnett A J, Zalesky A and Fitzgerald P B 2019 A multivariate neuroimaging biomarker of individual outcome to transcranial magnetic stimulation in depression Hum. Brain Mapp. 40 4618-29
17. Groppa S. Werner-Petroll N, Miinchau A, Deuschl Ruschworth M F and Siebner H R 2012 A novel dual-site transcranial magnetic stimulation paradigm to probe fast facilitatory inputs from ipsilateral dorsal premotor cortex to primary motor cortex Neuroimage 62 500-9
18. Parmigiani S, Barchiesi G and Cattaneo L 2015 The dorsal premotor cortex exerts a powerful and specific inhibitory effect on the ipsilateral corticofacial system: a dual-coil transcranial magnetic stimulation study Exp. Brain Res. 233 3253-60
19. Meng Q, Daugherty M, Patel P, Trivedi S, Du X, Hong E, et al. 2018 High-sensitivity and spatial resolution transient magnetic and electric field probes for transcranial magnetic stimulator characterizations. In-strum Sci Technol 46 502-18.
20. Deng Z-D, Lisanby S H, Peterchev A V. 2013 Electric field depth-focality tradeoff in transcranial magnetic stimulation: simulation comparison of 50 coil designs. Brain Stimulat 6 1-13.
21. Roth B J, Saypol J M, Hallett M, Cohen L G. 1991 A theoretical calculation of the electric field induced in the cortex during magnetic stimulation. Electroencephalogr Clin Neurophysiol Potentials Sect 81 47-56.
22. Gomez, L J, Goetz, S M, Peterchev, A V. 2018 Design of transcranial magnetic stimulation coils with optimal trade-off between depth, focality, and energy. J Neur Eng 15 046033.

What is claimed is:

1. An angled-tuned (AT) transcranial magnetic stimulation (TMS) coil device comprising:
a non-metal coil holder comprising a coil holder inner diameter and a coil holder outer diameter, wherein the coil holder inner diameter defines a hollow core;
at least two winding layers, wherein the at least one winding layer comprises wire wrapped around the coil holder outer diameter, wherein the width and height of each winding layer is in a range from about 0.5-5 mm and about 0.5 mm-2.5 cm, respectively; and
a separating layer between each winding layer,
wherein the at least two winding layers are substantially parallel to one another and are arranged at an angle of about 0° to about 80° relative to a horizontal plane of the non-metal coil holder.

2. The AT coil device of claim 1, wherein the non-metal coil holder is monolithic.

3. The AT coil device of claim 1, wherein the separating layer(s) comprise the same material as the non-metal coil holder.

4. The AT coil device of claim 1, wherein the hollow core further comprises a core material that is different from the material of the non-metal coil holder.

5. The AT coil device of claim 4, wherein the core material is selected from the group consisting of ferromagnetic materials, iron, cobalt, and nickel.

6. The AT coil device of claim 1, wherein the angle of the at least two winding layers is in a range from about 10° to about 80° relative to the horizontal plane of the non-metal coil holder.

7. The AT coil device of claim 1, wherein the coil holder outer diameter is in a range from about 1 cm to about 40 cm.

8. The AT coil device of claim 1, wherein the wire is litz wire.

9. A TMS system comprising:
a mechanical frame; and
at least one AT coil device of claim 1 attached to the mechanical frame for adjustment of the at least one AT coil device in the TMS system.

10. The TMS system of claim 9, wherein the hollow core further comprises a core material that is different from the material of the non-metal coil holder.

11. The TMS system of claim 10, wherein the core material is selected from the group consisting of ferromagnetic materials, iron, cobalt, and nickel.

12. The TMS system of claim 9, wherein the angle of the at least two winding layers of the AT coil device is in a range from about 10° to about 80° relative to the horizontal plane of the non-metal coil holder.

13. The TMS system of claim 9, wherein the TMS system comprises 2, 3, 4, 5, 6, 7, or 8 AT coil devices, wherein the AT coil devices are the same as or different from one another.

14. The TMS system of claim 13, comprising a pair of AT coil devices arranged in a “V shape,” relative to a horizontal plane.

15. The TMS system of claim 13, comprising a pair of AT coil devices arranged in a “A shape,” relative to a horizontal plane, to form an elliptical beam pair.

16. The TMS system of claim 15, wherein two elliptical beam pairs are arranged in a “V arrangement” to form a composite 4-coil structure.

17. The TMS system of claim 15, wherein four elliptical beam pairs are arranged in two “V arrangements” to form a composite 8-coil structure.

18. The TMS system of claim 16, wherein the angle of the two elliptical beam pairs in the “V arrangement” is in a range from about 30° to 70°.

19. The TMS system of claim 17, wherein the angle of the two elliptical beam pairs in the “V arrangement” is in a range from about 30° to 70°.

20. The TMS system of claim 15, wherein angle between each AT coil device in the elliptical beam pair is in a range from about 5° to 45°.
